(19) United States
(12) Patent Application Publication
(10) Pub. No.: US 2014/0162899 A1
(43) Pub. Date: Jun. 12, 2014

(54) CANCER PROGRESSION OBSERVATION INDEX GENE GROUP AND METHOD OF DETECTING THE GENE GROUP

(71) Applicants: Hee Sun Kim, Seoul (KR); Seung Jin Choi, Seoul (KR); Seok Chul Shin, Seoul (KR)

(72) Inventors: Hee Sun Kim, Seoul (KR); Seung Jin Choi, Seoul (KR); Seok Chul Shin, Seoul (KR)

(73) Assignee: KOREA HYDRO & NUCLEAR POWER CO., LTD., Gyeongju-si, Gyeongsangbuk-do (KR)

(21) Appl. No.: 14/239,247

(22) PCT Filed: Aug. 17, 2011
(86) PCT No.: PCT/KR2011/006025

§ 371 Date: Feb. 24, 2014

(51) Int. Cl. C12Q 1/68

(52) U.S. Cl. CPC C12Q 1/6886
USPC 506/9; 435/6.14; 506/17

(57) ABSTRACT

Provided are a cancer progression observation index gene group and a method of detecting the gene group. The gene group includes cancer progression observation index genes increased or decreased when high- or low-dose radiation is performed on a mouse in which an oncogene is inserted into a thymocyte, for example, Itgb3 and Igf1 increased due to low-dose radiation to suppress the conversion of the thymocyte into a cancer cell, and Itga4, Itgb1, Itgav, Itga6, Itgb4, Raf, Myc, Fos, Trp53 and Apaf1 decreased due to high-dose radiation to stimulate the conversion of the thymocyte into a cancer cell. The gene group and the method may be used to clearly define a cancer progression observation index specifically responding to radiation.

### CROSS-REFERENCE TO RELATED APPLICATIONS

This application is a national phase application of International Application No. PCT/KR2011/006025, filed Aug. 17, 2011. All disclosures of the document(s) named above are incorporated herein by reference.

### BACKGROUND OF THE INVENTION

1. Field of the Invention

The present invention relates to a cancer progression observation index gene group and a method of detecting the gene group, and more particularly, to an observation index gene capable of confirming cancer progression found in common among AKR/J mice exposed to high- and low-dose ionizing radiation and a method of detecting the observation index gene.

2. Description of the Related Art

In general, there are various studies on an increase in application of radiation to medical and general industries and influence of radiation on a human body. Particularly, the majority of such studies are about a relationship between radiation and cancer.

It is known that ionizing radiation causes an increase in dose and diseases including cancer and deformities, but radiation having a dose of 200 mGy or less or 6 mGy/hr or less recovers a damaged gene and suppresses the occurrence of cancer.

However, since the conventional study on the relationship between radiation and the occurrence of cancer changes a part of a gene or uses a cancer cell line, various responses observed in the cell, tissue, organ and body constituting an entire body could not be explained. To compensate for such a disadvantage, studies on occurrence of cancer with respect to radiation are performed using mice in which 95% or more of genes are similar to those of a human.

However, since a general mouse has very low natural occurrence of cancer, various model mice for cancer studies are used.

Particularly, studies on immune gene responses have been conventionally performed, but many confounding variables suppressing reliability of the results have interfered. This is because most of the conventional studies used cancer cells or general mice, and thus the expressed immune gene was fragmented and difficult to apply to an individual.

A method using a cell for the conventional cancer studies included changing an immune gene or performing radiation on a p53-deficient cancer cell which was important in the occurrence of cancer, and a basic response of the cancer cell was different from that of a normal cell. Thus, the result could not be applied to an individual. In addition, since the conventional method evaluated an immune gene response using a general mouse, various genes were expressed, and cancer did not occur in a specific organ, it was difficult to interpret a gene response.

In addition, in Korean Unexamined Patent Application 10-2004-0103677, a marker capable of diagnosing cell damage according to low- or high-dose radiation is disclosed. However, the application does not focus on a dose, and also includes a method including high-dose radiation after low-dose radiation.

### SUMMARY OF THE INVENTION

The present invention is directed to providing a cancer progression observation index gene group detected in a mouse exposed to ionizing radiation capable of ensuring a gene profile specifically responding to radiation on an individual level, and a method of detecting the observation index mouse.

One aspect of the present invention provides a cancer progression observation index gene group, which includes cancer progression observation index genes increased or decreased when high- or low-dose radiation is applied to a mouse in which an oncogene is inserted into a thymocyte, for example, Itgb3, Igf1, Itga4, Itgb1, Itgav, Itga6, Itgb4, Raf, Myc, Fos, Trp53 and Apaf1. Among these genes, Itgb3 and Igf1 are decreased or increased to suppress conversion of the thymocyte into a cancer cell, and Itga4, Itgb1, Itgav, Itga6, Itgb4, Raf, Myc, Fos, Trp53 and Apaf1 are decreased in high-dose radiation to stimulate conversion of the thymocyte into a cancer cell.

Another aspect of the present invention provides a method of detecting the cancer progression observation index gene group, which includes a) preparing a plurality of AKR/J mice in which an oncogene is inserted into a thymocyte, b) dividing the AKR/J mice into three groups, raising the first group of AKR/J mice after high-dose ionizing radiation, raising the second group of AKR/J mice after low-dose ionizing radiation, and raising the third group of AKR/J mice in a general environment, c) obtaining a thymus of a dead mouse of the first or second group of the AKR/J mice throughout the operation b) and diagnosing cancer when a weight of the thymus is increased to twice or more that before radiation, d) extracting thymuses by sacrificing the first to third groups of the AKR/J mice at the time when the third group of the AKR/J mice initially die, and e) selecting only a thymocyte having no change in weight compared to the organs of the same-aged non-irradiated AKR/J mice from the extracted organs and detecting a gene of the organ whose weight is increased or decreased by a factor of two or more through gene analysis.

Additional aspects and/or advantages of the invention will be set forth in part in the description which follows and, in part, will be obvious from the description, or may be learned by practice of the invention.

### BRIEF DESCRIPTION OF THE DRAWINGS

The above and other objects, features and advantages of the present invention will become more apparent to those of ordinary skill in the art by describing in detail exemplary embodiments thereof with reference to the adhered drawings, in which:

FIG. 1 is a flowchart of detecting a cancer progression observation index gene group detected from a mouse exposed to ionizing radiation according to an exemplary embodiment of the present invention;

FIG. 2 is a graph showing an accumulated value of dead mice caused by the occurrence of thymic carcinoma in each test group on a percentage basis;

FIG. 3 is a diagram of an expression mechanism of cancer progression observation index genes in a low-dose irradiated AKR/J mouse which have an influence on the occurrence of cancer; and

FIG. 4 is a diagram of an expression mechanism of cancer progression observation index genes in a high-dose irradiated AKR/J mouse which have an influence on the occurrence of cancer.

### DETAILED DESCRIPTION OF EXEMPLARY EMBODIMENTS

Hereinafter, exemplary embodiments of the present invention will be described in detail. However, the present invention is not limited to the embodiments disclosed below, but can be implemented in various forms. The following embodiments are described in order to enable those of ordinary skill in the related art to embody and practice the present invention.

Although the terms first, second, etc. may be used to describe various elements, these elements are not limited by these terms. These terms are only used to distinguish one element from another. For example, a first element could be termed a second element, and, similarly, a second element could be termed a first element, without departing from the scope of exemplary embodiments. The term “and/or” includes any and all combinations of one or more of the associated listed items.

The terminology used herein is for the purpose of describing particular embodiments only and is not intended to be limiting of exemplary embodiments. The singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, components and/or groups thereof, but do not preclude the presence or addition of one or more other features, integers, steps, operations, elements, components and/or groups thereof.

With reference to the appended drawings, exemplary embodiments of the present invention will be described in detail below. To aid in understanding the present invention, like numbers refer to like elements throughout the description of the figures, and the description of the same elements will be not reiterated.

Hereinafter, a cancer progression observation index gene group detected from a mouse exposed to ionizing radiation constituted as described above will be described in detail with reference to the accompanying drawings.

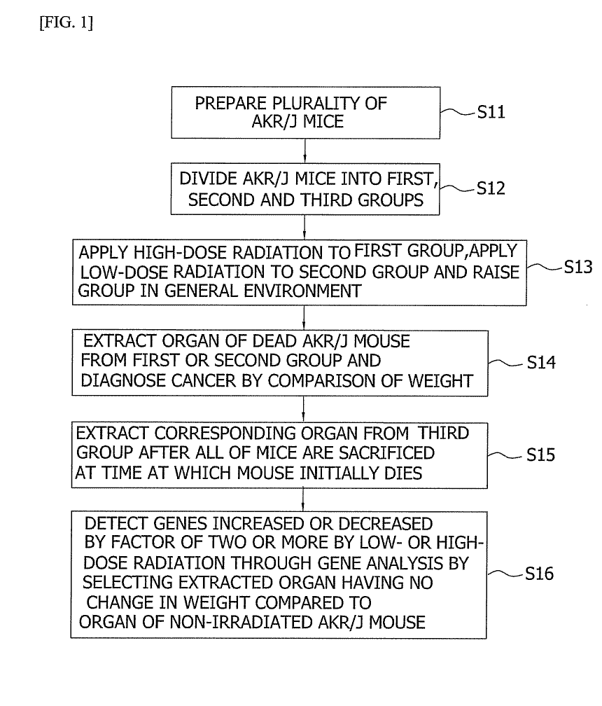

FIG. 1 is a flowchart of detecting a cancer progression observation index gene group detected from a mouse exposed to ionizing radiation according to an exemplary embodiment of the present invention.

Referring to FIG. 1, the process includes preparing a plurality of AKR/J mice in which an oncogene is inserted into a thymocyte (S11), dividing the AKR/J mice into a first group subjected to high-dose ionizing radiation, a second group subjected to low-dose ionizing radiation, and a third group not subjected to ionizing radiation (S12), performing high-dose ionizing radiation on the first group of the AKR/J mice, and low-dose ionizing radiation on the second group of the AKR/J mice (S13), obtaining a thymus of a dead mouse of the first or second group of the AKR/J mice and diagnosing cancer when a weight of the thymus is increased to twice or more that before radiation (S14), extracting thymuses by sacrificing the first to third groups of the AKR/J mice at the time at which the third group of the AKR/J mice initially die (S15), and selecting only a thymocyte having no change in weight compared to the organs of the same-aged non-irradiated AKR/J mice from the extracted organs and obtaining DNA recovery, a DNA damage signal, a cell cycle, a cancer progression observation index, a p53 signal system transduction pathway, and apoptosis T- and B-cell activation gene profiles through gene analysis (S16).

Hereinafter, the method of detecting a cancer progression observation index gene group according to the exemplary embodiment of the present invention described above will be described in further detail.

First, in S11, AKR/J mice in which thymic carcinoma naturally occurs according to time due to a mouse leukemia virus gene fragment inserted into a thymocyte chromosome DNA are prepared.

The AKR/J mice used in the test are 6-week old females manufactured by SLC, Japan.

Such AKR/J mice are divided into three groups, and in S13, the first group is subjected to high-dose ionizing radiation, the second group is subjected to low-dose ionizing radiation, and the third group is raised without radiation.

For high-dose ionizing radiation to the first group, gamma rays (Cs-137) are radiated at a dose of 0.8 Gy/min to have a final dose of 4.5 Gy using a gamma ray generating device (IBL 147C, CIS bio-international, France).

In addition, the second group is irradiated to have an accumulated dose of 4.5 Gy in an environment in which gamma rays (Cs-137) having a low dose of 0.7 mGy/hr are radiated, and the third group of the AKR/J mice are raised in a general environment in which no radiation is applied.

Afterward, in S14, when the AKR/J mice included in the first to third groups die during S13, thymuses are obtained from the dead mice by autopsy.

Here, when a weight of an obtained thymus is twice or more an average weight of a thymus of the same aged other AKR/J mouse which is not irradiated, it is determined that thymic carcinoma occurs.

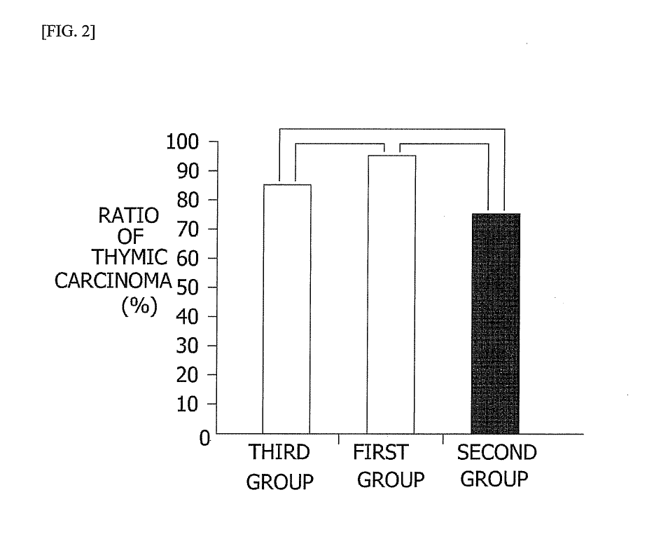

FIG. 2 is a graph showing an accumulated value of dead mice caused by the occurrence of thymic carcinoma in each test group on a percentage basis.

Referring to FIG. 2, it can be seen that the second group of the low-dose irradiated mice have lower occurrence of thymic carcinoma than the first group of the high-dose irradiated mice.

Afterward, in S15, based on the time at which the third group of the AKR/J mice initially die, all of the AKR/J mice are sacrificed, and a thymus is obtained from each AKR/J mouse.

The third group of the AKR/J mouse raised in a general environment also have a cancer-occurring gene inserted into a thymocyte, and die after a predetermined time because of thymic carcinoma. Experimentally, it was found that the first mouse died after 150 days of the experiment.

The process in which the thymic carcinoma occurs is fixed to prevent interference of confounding variables.

Afterward, in S16, only thymuses having no weight change of the obtained thymuses are immersed in liquid nitrogen, and gene analysis is performed.

Here, the gene analysis method includes crushing a thymocyte of the obtained AKR/J mouse and performing detection using a microarray kit manufactured by Agilent, and thus is used to analyze expression of a cancer progression observation index genes which are expressed increased or decreased by a factor of two or more in the second group subjected to low-dose radiation.

Table 1 shows digitization of relative change in genes of a mouse in the second group subjected to low-dose radiation when a chip is scanned after the microarray test, compared to genes of a non-irradiated mouse of the second group. Here, it can be confirmed that, for Igf, a ratio to non-irradiation is increased by a factor of 2.1, and for Itgb3, a ratio to non-irradiation is decreased by a factor of 0.2.

SSS

TABLE 1

| | Gene Bank | | Ratio to |
|---|---|---|---|
| Gene | Accession No. | Name | non-irradiation |
| Igf1 | NM_184052 | Insulin-like growth | 2.1 |
| | | factor1 | |
| Itgb3 | NM_016780 | Intergrin beta 3 | 0.2 |

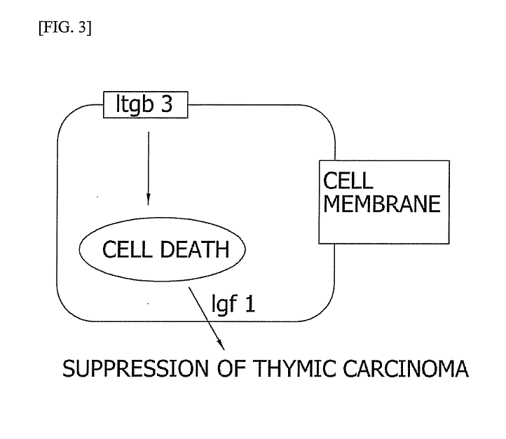

FIG. 3 is a diagram of an expression mechanism of cancer progression observation index genes in a low-dose irradiated AKR/J mouse which have an influence on the occurrence of cancer.

Referring to FIG. 3, progression of the thymocyte into which a gene initiating the occurrence of cancer is inserted to cancer is suppressed in the low-dose irradiated AKR/J mouse. This is because the death of a cancer cell is induced according to a decrease in an Itgb3 gene in the cancer-progressing cell.

In addition, as the Igf1 gene is increased, survival of a normal T-cell is stimulated, damaged DNA is recovered, and the change of the thymocyte into cancer is suppressed. Such a mechanism actively occurs to prevent the progression of the thymocyte into the cancer and prolong a life span.

Afterward, a gene expressed by a factor of two times or more in the thymus extracted from the first group of the high-dose irradiated mice is analyzed.

Table 2 shows digitization of relative increase in genes of the high-dose irradiated mouse when a chip is scanned after the microarray test, compared to a non-irradiated group.

Specifically, it can be confirmed that Cds1 is increased by a factor of 3.6, Vegfb is increased by a factor of 3.2, Itgb3 is increased by a factor of 2.6, and S100a4 and Gzma are increased by a factor of 2.2.

TABLE 2

| | Gene Bank | | Ratio to |
|---|---|---|---|
| Gene | Accession No. | Name | non-irradiation |
| Cds1 | NM_173370 | CDP-diacylglycerol | 3.6 |
| | | synthase1 | |
| Vegfb | NM_011697 | Vascular endothelial | 3.2 |
| | | growth factor B | |
| Itgb3 | NM_016780 | Integrin beta 3 | 2.6 |
| S100a4 | NM_011311 | S100 calcium binding | 2.2 |
| | | protein A4 | |
| Gzma | NM_010370 | Granzyme A | 2.2 |

In addition, expression of genes decreased by a factor of two or less in the thymuses extracted from the first group of the high-dose irradiated mice is analyzed.

Table 3 shows digitization of a relative decrease in genes of the high-dose irradiated mouse when a chip is scanned after the microarray test, compared to a non-irradiated group.

TABLE 3

| | Gene Bank | | Ratio to |
|---|---|---|---|
| Gene | Accession No. | Name | non-irradiation |
| Myc | NM_010849 | Myelocytomatosis | 0.5 |
| | | oncogene | |
| Apaf1 | NM_009684 | Apoptotic peptidase | 0.5 |
| | | activating factor1 | |
| cdc25a | NM_007658 | Cell division cycle 25 | 0.49 |
| | | homolog A | |
| | | (S. cerevisiae) | |
| Itga4 | NM_010576 | Integrin alpha 4 | 0.48 |
| Mta1 | NM_054081 | Metastasis | 0.38 |
| | | associated 1 | |
| Itga6 | BC024571 | Integrin alpha 6 | 0.11 |
| Itgav | BC048857 | Integrin alpha V | 0.37 |
| Fos | NM_010234 | FBJ osteosarcoma | 0.36 |
| | | oncogene | |
| Trp53 | NM_011640 | Transformation | 0.34 |
| | | related protein 53 | |
| Raf1 | AK036317 | V-raf-leukemia viral | 0.27 |
| | | oncogene 1 | |
| Pten | AK037998 | Phosphatase and | 0.26 |
| | | tensin homolog | |
| Akt2 | NM_007434 | Thymoma viral proto- | 0.25 |
| | | oncogene 2 | |
| Pik3r1 | NM_001024955 | Phosphatidylinositol | 0.24 |
| | | 3-kinase, regulatory | |
| | | subunit, polypeptide | |
| | | 1(p85 alpha) | |
| Itgb1 | NM_010578 | Integrin beta | 0.19 |
| | | 1(fibronectin receptor | |
| | | beta) | |
| Mdm2 | NM_010786 | Transformed mouse | 0.38 |
| | | 3T3 cell double | |
| | | minute 2 | |
| Cdk2 | NM_183417 | Cyclin-dependent | 0.09 |
| | | kinase 2 | |

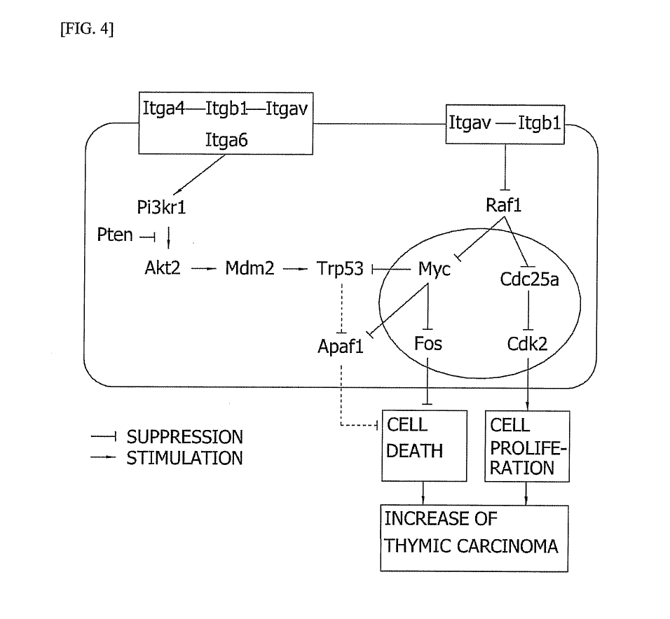

FIG. 4 is a diagram of an expression mechanism of cancer progression observation index genes in a high-dose irradiated AKR/J mouse, which have an influence on the occurrence of cancer.

Referring to FIG. 4, Itga4, Itgb1, Itgav, Itga6 and Itgb4 form an integrin complex receptor on a surface of a cell membrane of the thymocyte of the high-dose irradiated AKR/J mouse to serve as an oncogene.

In addition, Raf, Myc, Fos, Trp53 and Apaf1 genes removing damaged cells by death are considerably suppressed to prevent the death of the damaged thymocyte and stimulate the occurrence of thymic carcinoma.

On the other hand, the Pi3kr1 gene stimulating cell death and the Akt2 and Mmd2 genes are activated, but Trp53 is suppressed, thereby preventing the cell death. It can be seen that as Cdc25a and Cdk2 of the genes relating to a cell cycle are repressed, the damaged DNA is not recovered and proliferation of damaged thymic carcinoma cells is stimulated, thereby stimulating progression to thymic carcinoma.

According to the present invention composed as described above, a cancer progression observation index gene specifically responding to radiation can be clearly defined by fixing the occurrence of cancer by obtaining a thymus at the time at which an AKR/J mouse in which an oncogene is inserted into the thymocyte to serve as a thymic carcinoma initiation factor, and preventing the interference of confounding variables to interpret a gene response within the thymuses having no change in weight compared to a non-irradiated mouse thymus.

Such an observation index gene serves to diagnose the occurrence of thymic carcinoma and predict progression of cancer, and thus can be used as a kit for diagnosing cancer.

The present invention relates to a cancer progression observation index gene group capable of confirming progression of cancer and a method of detecting the gene group, and a diagnosing kit capable of confirming the progression of cancer can be developed.

While the invention has been shown and described with reference to certain exemplary embodiments thereof, it will be understood by those skilled in the art that various changes in form and details may be made therein without departing from the scope of the invention as defined by the appended claims.

1. A method of detecting a cancer progression observation index gene group, comprising:
a) preparing a plurality of AKR/J mice in which an oncogene is inserted into a thymocyte;
b) dividing the AKR/J mice into three groups, raising the first group of the AKR/J mice after high-dose ionizing radiation, raising the second group of the AKR/J mice after low-dose ionizing radiation, and raising the third group of the AKR/J mice in a general environment;
c) obtaining a thymus of a dead mouse of the first or second group of the AKR/J mice throughout the b) operation and diagnosing cancer when a weight of the thymus is increased to twice or more that before radiation;
d) extracting thymuses by sacrificing the first to third groups of the AKR/J mice at the time at which the third group of the AKR/J mice initially die; and
e) selecting only a thymocyte having no change in weight compared to the organs of the same-aged non-irradiated AKR/J mice from the organs extracted in d) and detecting a gene of the organ whose weight is increased or decreased by a factor of two or more through gene analysis.

2. The method according to claim 1, wherein, in b), gamma rays (Cs-137) are radiated onto the first group at a dose of 0.8 Gy/min to have a final dose of 4.5 Gy, and low-dose gamma rays (Cs-137) are radiated onto the second group to have an accumulated dose of 4.5 Gy.

3. The method according to claim 1, wherein, in e), the detected gene group is used as an index for manufacturing a thymic carcinoma diagnosing kit, evaluating cancer progression and the extent of treatment, evaluating radiation exposure and relevance to the occurrence of cancer of an industrial and medical employee, evaluating a causal relationship between radiation and the occurrence of cancer, evaluating a biological dose with respect to radiation exposure, or evaluating an effect of suppressing thymic carcinoma by low-dose radiation.

4. The method according to claim 1, wherein, in e), a gene killing a damaged cell by reducing low-dose radiation to suppress the occurrence of cancer is Itgb3.

5. The method according to claim 1, wherein, in e), a gene stimulating the survival of a normal T-cell by increasing low-dose radiation, recovering damaged DNA, and suppressing the change of the thymocyte into cancer is Igf1.

6. The method according to claim 1, wherein, in e), genes decreased due to high-dose radiation to stimulate the change of a thymocyte into cancer are Itga4, Itgb1, Itgav, Itga6 and Itgb4, which form an integrin complex receptor to serve as an oncogene.

7. The method according to claim 1, wherein, in e), genes decreased due to high-dose radiation to suppress the death of a damaged cell and stimulate the occurrence of thymic carcinoma are Raf, Myc, Fos, Trp53 and Apaf1.

8. A cancer progression observation index gene group including cancer progression observation index genes increased or decreased when high- or low-dose radiation is performed on a mouse in which an oncogene is inserted into a thymocyte,
wherein the genes decreased or increased by low-dose radiation to convert the thymocyte into a cancer cell are Itgb3 and Igf1, and the genes decreased by high-dose radiation to stimulate the conversion of the thymocyte into a cancer cell are Itga4, Itgb1, Itgav, Itga6, Itgb4, Raf, Myc, Fos, Trp53 and Apaf1.

9. The method according to claim 2, wherein, in e), the detected gene group is used as an index for manufacturing a thymic carcinoma diagnosing kit, evaluating cancer progression and the extent of treatment, evaluating radiation exposure and relevance to the occurrence of cancer of an industrial and medical employee, evaluating a causal relationship between radiation and the occurrence of cancer, evaluating a biological dose with respect to radiation exposure, or evaluating an effect of suppressing thymic carcinoma by low-dose radiation.

10. The method according to claim 2, wherein, in e), a gene killing a damaged cell by reducing low-dose radiation to suppress the occurrence of cancer is Itgb3.

11. The method according to claim 2, wherein, in e), a gene stimulating the survival of a normal T-cell by increasing low-dose radiation, recovering damaged DNA, and suppressing the change of the thymocyte into cancer is Igf1.

12. The method according to claim 2, wherein, in e), genes decreased due to high-dose radiation to stimulate the change of a thymocyte into cancer are Itga4, Itgb1, Itgav, Itga6 and Itgb4, which form an integrin complex receptor to serve as an oncogene.

13. The method according to claim 2, wherein, in e), genes decreased due to high-dose radiation to suppress the death of a damaged cell and stimulate the occurrence of thymic carcinoma are Raf, Myc, Fos, Trp53 and Apaf1.
